(19) United States
(12) Patent Application Publication
(10) Pub. No.: US 2009/0230169 A1
(43) Pub. Date: Sep. 17, 2009

(54) Endostapler Biasing Mechanism

(75) Inventors: Jia Hua Xiao, Santa Rosa, CA (US); Eric Meyer, Andover, MN (US); Jeffrey Sandstrom, Forest Lake, MN (US); Damian Jelich, Cottage Grove, MN (US); Trevor Greenan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/407,495

(22) Filed: Mar. 19, 2009

Related U.S. Application Data
(63) Continuation-in-part of application No. 12/049,531, filed on Mar. 17, 2008.

(51) Int. Cl. A61B 17/068

(52) U.S. Cl. 227/175.1

(57) ABSTRACT

An endostapler delivery system includes a biasing mechanism to offset or counter forces generated by a stapling device and therefore prevent the stapling device from moving during the firing of the staple. The delivery system includes a catheter having at least one lumen extending there through for receiving the stapling device. The biasing mechanism is an expandable biasing cage having a dome or semi-circular expanded shape provided at the distal portion of the catheter. When expanded, the biasing cage does not block or occlude a vessel, thereby allowing blood flow to continue during the stapling procedure. The endostapler delivery system further includes a steering wire that can be used to bend the catheter shaft.

### CROSS-REFERENCE TO RELATED APPLICATIONS

This application is a continuation-in-part of U.S. application Ser. No. 12/049,531 filed Mar. 17, 2008, the entirety of which is incorporated by reference herein.

### FIELD OF THE INVENTION

The present invention relates generally to endostapler delivery systems employed in the treatment of vascular disease. More particularly, the present invention relates to endostapler delivery systems including a biasing mechanism for use in the fixation of grafts to the walls of vessels.

### BACKGROUND

In modern medical practice, it is sometimes desirable to pass a stapling device into or through the wall of a luminal anatomical structure (e.g., a blood vessel or other anatomical conduit) for the purpose of attaching an article (e.g., an endoluminal, extraluminal or transluminal graft) or other apparatus to the wall of the anatomical structure.

Examples of medical procedures wherein it is desirable to anchor or attach a graft or other apparatus to the wall of a blood vessel or other luminal anatomical conduit include certain endovascular grafting procedures wherein a tubular graft is placed within the lumen of an aneurysmal blood vessel to create a neo-lumen or artificial flow conduit through an aneurysm, thereby reducing if not completely eliminating the exertion of blood pressure on the aneurysm and allowing the aneurysmal sac to subsequently become stagnant and transform to granulation tissue. These endovascular grafting procedures have heretofore been used to treat aneurysms of the abdominal aorta, as well as aneurysms of the descending thoracic aorta. Endovascular grafts used typically incorporate or are combined with one or more radially expandable stents which are radially expanded in situ to anchor the tubular graft to the wall of the blood vessel at sites upstream and downstream of the aneurysm. Thus, the grafts are typically held in place by mechanical engagement, tissue ingrowth, and friction via the self-expanding or balloon expandable stents. The grafts may also be affixed to vessels with hooks or barbs.

However, in the event that the force provided by these stent(s) fails to establish sound mechanical and/or frictional engagement with the blood vessel wall, the graft may undergo undesirable migration or slippage, or blood may leak into the aneurysmal sac (sometimes referred to as an “endoleak”). Thus, in view of the above-mentioned undesirable complications associated with the use of radially expandable stents to mechanically and/or frictionally anchor a graft or other apparatus to the wall of a blood vessel (or other luminal anatomical structure) there exists a need in the art for the development of new endoluminal attachment devices which may be used to attach the ends of a endoluminal tube graft (or other article) to the surrounding wall of a blood vessel or other tubular anatomical conduit, thereby ensuring sound and permanent placement of the graft or other article.

### SUMMARY OF THE INVENTION

Embodiments described herein relate to an endostapler delivery system for delivering a stapling device through a body lumen. The system includes a catheter shaft including a proximal portion and a distal portion, the catheter shaft defining a first lumen having a first exit port disposed at the distal portion of the catheter shaft and a second lumen having a second, side exit port disposed at the distal portion of the catheter shaft. The first lumen of the catheter shaft is of a sufficient size such that the stapling device may be advanced there through. An expandable biasing cage is disposed within the second lumen of the catheter shaft. A first actuator is disposed at the proximal portion of the catheter shaft, wherein the actuator is adapted to expand the biasing cage to a dome shape extending outside of the catheter shaft via the second, side exit port such that the biasing cage abuts a vessel wall of the body lumen and/or a graft implanted within the body lumen. The biasing cage when expanded does not block or occlude the body lumen such that blood may flow there through. A steering wire is also disposed in the second lumen and is coupled at its distal end to the biasing cage and at is proximal end to a second actuator. Operating the second actuator pulls the steering wire to bend the catheter shaft to steer the catheter and/or to provide apposition for the stapler when the stapler is used in a curved or angled portion of a vessel.

### BRIEF DESCRIPTION OF DRAWINGS

The foregoing and other features and advantages will be apparent from the following description of embodiments as illustrated in the accompanying drawings. The accompanying drawings, which are incorporated herein and form a part of the specification, further serve to explain the principles used in the embodiments. The drawings are not to scale.

FIG. 1 is a schematic isometric view of an endostapler delivery system.

FIG. 2 is a cross-sectional view of a vessel within which the endostapler delivery system in FIG. 1 (only the end of which can be seen) is configured to position the stapler opening of the system adjacent the vessel wall for attaching an endoluminal graft to a vessel wall.

FIG. 3 is a sectional side view of the endostapler delivery system of FIG. 1, wherein a ribbon of a biasing cage of the endostapler delivery system is in an unexpanded configuration.

FIG. 4 is a sectional side view of the endostapler delivery system of FIG. 1, wherein the ribbon of the biasing cage of the endostapler delivery system is in an expanded configuration.

FIG. 5A is a cross-sectional view of the endostapler delivery system of FIG. 1.

FIG. 5B is a cross-sectional view of another embodiment of the endostapler delivery system of FIG. 1.

FIG. 6 is a pictorial view of a distal portion of the endostapler delivery system illustrated in FIG. 1, wherein the biasing cage of the endostapler delivery system in an expanded configuration.

FIG. 7 is a schematic isometric view of another embodiment of an endostapler delivery system.

FIG. 8 is a cross-sectional view of a vessel within which the endostapler delivery system in FIG. 7 (only the end view of which can be seen) is configured to position the stapler opening of the system adjacent the vessel wall.

FIG. 9A is a sectional side view of the endostapler delivery system of FIG. 7, wherein a plurality of braided elements of a biasing cage of the endostapler delivery system are in an unexpanded configuration.

FIG. 9B is a sectional side view of the endostapler delivery system of FIG. 7, wherein the braided elements of the biasing cage of the endostapler delivery system are in an expanded configuration.

FIG. 10 is a side pictorial view of a distal portion of the endostapler delivery system of FIG. 7, wherein the braided elements of the biasing cage of the endostapler delivery system are in an expanded configuration.

FIG. 11 is a schematic isometric view of another embodiment of an endostapler delivery system.

FIG. 12 is a cross-sectional view of a vessel within which the endostapler delivery system of FIG. 11 (only the end view of which can be seen) is configured to position the stapler opening of the system adjacent the vessel wall.

FIG. 13A is a sectional side view of the endostapler delivery system of FIG. 11, wherein the braided elements and ribbon of a biasing cage of the endostapler delivery system are in an unexpanded configuration.

FIG. 13B is a sectional side view of the endostapler delivery system of FIG. 11, wherein the braided elements and ribbon of a biasing cage of the endostapler delivery system are in an expanded configuration.

FIG. 14 is a top pictorial view of a distal portion of the endostapler delivery system of FIG. 11, wherein the braided elements and ribbon of the biasing cage of the endostapler delivery system are in an expanded configuration.

FIG. 15 is a side view illustration of a distal portion of the endostapler delivery system of FIG. 11, wherein the braided elements and ribbon of the biasing cage of the endostapler delivery system are in an expanded configuration.

FIG. 16 is a sectional side view of an endostapler delivery system according to another embodiment, wherein a biasing cage of the endostapler delivery system is in an unexpanded configuration.

FIG. 17 is a schematic illustration of an endostapler delivery system in accordance with another embodiment.

FIG. 18 is a partial longitudinal cross-sectional view of the endostapler delivery system of FIG. 17.

FIG. 19 is a schematic illustration of the distal portion endostapler delivery system of FIG. 17 with a steering wire used to bend the catheter.

FIG. 20 is a cross-section taken along line A-A of FIG. 18.

FIG. 21 is an alternative embodiment of the cross-section taken alone line A-A of FIG. 18.

FIG. 22 is a sectional side view of a distal portion of the endostapler delivery system of FIG. 17.

### DETAILED DESCRIPTION

Specific embodiments are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the number of possible variations of embodiments according to the invention. Although the description of embodiments is in the context of treatment of blood vessels such as the coronary, carotid and renal arteries, the embodiments may also be used in any other body passageways where it is deemed useful.

Embodiments described relate to an endostapler delivery system having a biasing mechanism to offset or counter forces generated by a stapling device.

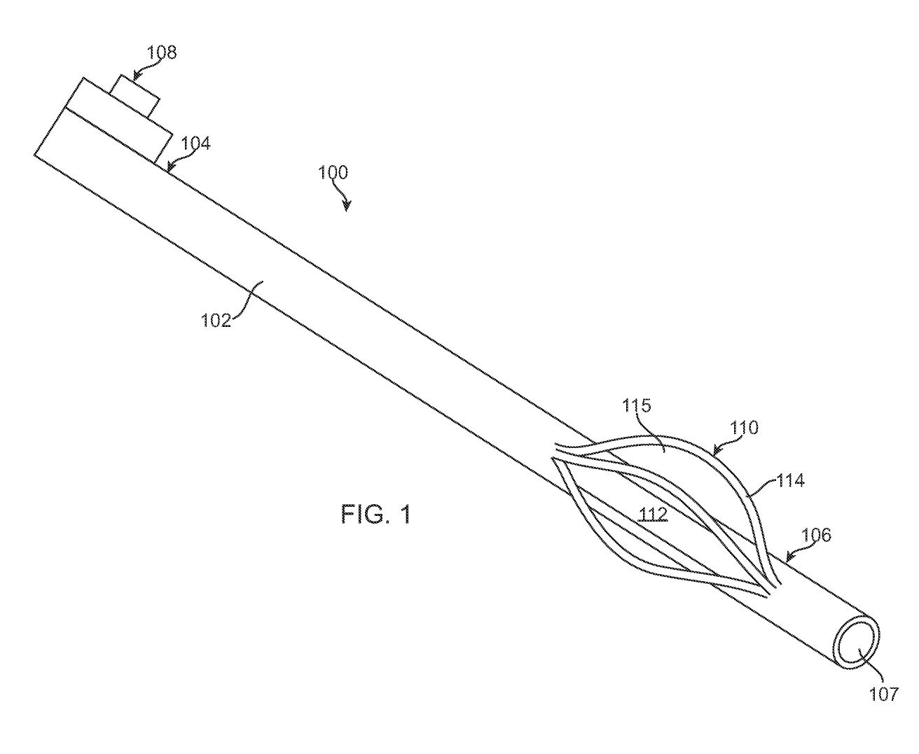

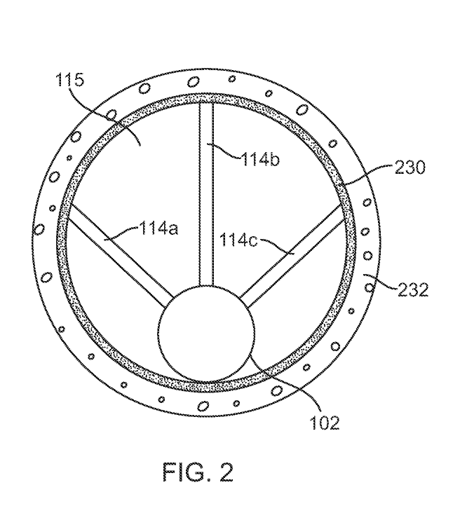

Referring to FIGS. 1-2, an endostapler delivery system 100 includes a catheter shaft 102 having an expandable biasing cage 110 disposed at the distal portion thereof. FIG. 1 is an schematic isometric view of endostapler delivery system 100, while FIG. 2 is an end view of the endostapler delivery system 100 positioned within a vessel for attaching an endoluminal graft to a vessel wall 232. Catheter shaft 102 includes a proximal portion 104 and a distal portion 106, wherein distal portion 106 includes an exit port 107. In addition, as will be explained in more detail below, catheter shaft 102 has at least one lumen extending there through for receiving a stapling device for attaching an endovascular graft 230 to a vessel wall 232 of a body lumen. A side recess or port 112 is provided at the distal portion 106 of catheter shaft 102 for exposing the expandable biasing cage 110. An actuator 108 is provided at the proximal portion 104 of catheter shaft 102 for expanding biasing cage 110 to a dome or semi-circular shape. Biasing cage 110 is expanded to the dome or semi-circular shape in situ in order to ensure that the stapling device abuts the vessel and/or graft. During operation of the stapling device, expanded biasing cage 110 acts as an anchor to offset or counter forces generated by the stapling device.

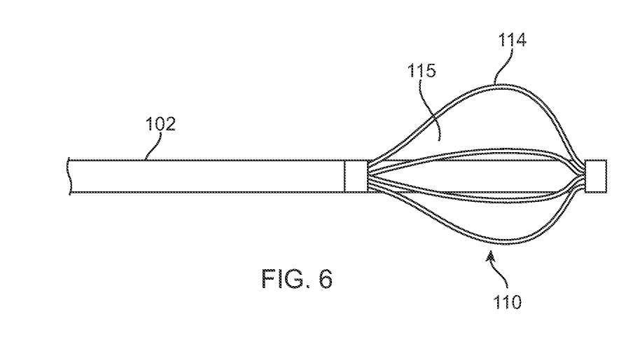

Biasing cage 110 includes a plurality of ribbons or strands 114 that extend generally parallel to the blood flow when expanded. Open spaces 115 disposed between the plurality of ribbons or strands 114 when biasing cage is expanded allow blood or other fluid to flow there through during the stapling procedure such that the blood vessel is not blocked or occluded. In one example shown in FIGS. 1-2 and 6, biasing cage 110 includes three ribbons 114a, 114b, and 114c. However, one of ordinary skill in the art will appreciate that biasing cage 110 may include any number of ribbons or strands. For example, biasing cage 110 may include between two and five ribbons or strands that extend generally parallel to the blood flow when expanded. The plurality of ribbons 114 have sufficient mechanical strength to anchor the catheter shaft 102 to offset or counter forces generated by a stapling device when the stapling device is utilized in securing endovascular graft 230 (only a cross section of which is shown) to a vessel wall 232 of a body lumen. More particularly, biasing cage 110 may be expanded prior to the firing of a staple. Expanding biasing cage 110 forces the stapling device against a receiving area of the vessel wall 232 and/or graft 230 where a staple is to be fired. Preferably, the receiving area of the vessel wall 232 and/or graft 230 is positioned on the opposite side of the vessel to the average centerline of force vectors associated with the expansion of the various components of the biasing cage 110. In addition to placing the stapling device immediately adjacent to the receiving area of the vessel wall 232 and/or graft 230, biasing cage 110 also assists with preventing the stapling device from moving during the firing of the staple.

Embodiments described may be used with any conventional stapling device capable of securing graft 230 to vessel wall 232. Thus, it will be apparent to those of ordinary skill in the art that any features of the stapling device discussed herein are exemplary in nature. For example, the stapling device may be any stapling device known in the art, including but not limited to those shown or described in US Patent Publication 20040176786 assigned to Edrich Vascular, US Patent Publication 20070073389 assigned to Aptus Endosystems, Inc., and US Patent Publication 20070162053 assigned to Anson Medical. In another embodiment (not shown), the stapling device may be an integral part of the biasing endostapler delivery system, i.e., formed as one integral piece within a lumen of the catheter.

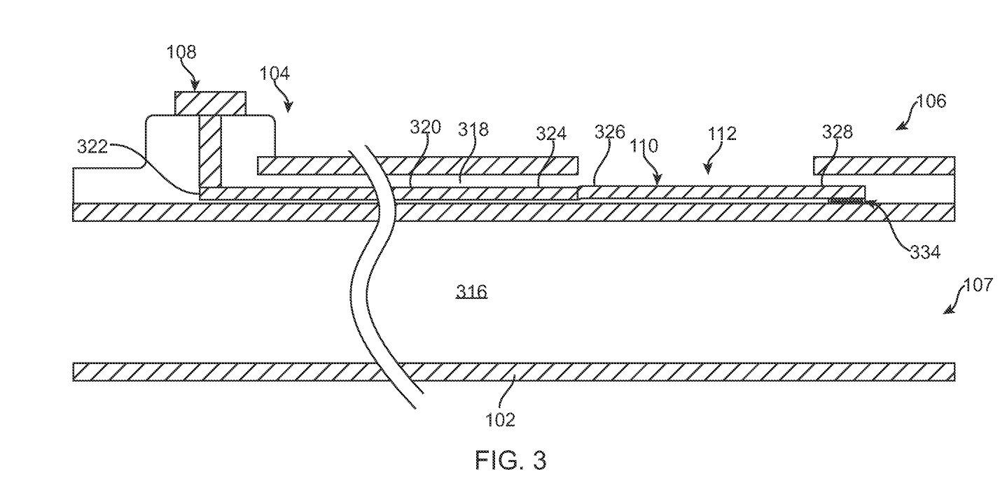

As shown in FIG. 3, catheter shaft 102 is a multi-lumen catheter. FIG. 3 is a sectional side view of the endostapler delivery system 100 illustrated in FIG. 1. Catheter shaft 102 includes a first lumen 316 extending along the entire length thereof for receiving a stapling device. In the present embodiment, first lumen 316 is open-ended and in fluid communication with exit port 107 such that the stapling device may exit out of the exit port 107 at the distal portion 106 of catheter shaft 102. However, as will be explained in greater detail herein, alternatively the first lumen may be closed-ended but in fluid communication with an exit port located in the side of the catheter shaft such that a side-firing stapling device may be used. Catheter shaft 102 also includes a second lumen 318 that extends from the proximal portion 104 to the distal portion 106 of catheter shaft 102 for housing the biasing mechanism, including biasing cage 110. Second lumen 318 is parallel and adjacent to first lumen 316. Second lumen 318 is closed-ended but in fluid communication with side recess or port 112 provided at the distal portion 106 of catheter shaft 102. Side recess or port 112 allows biasing cage 110 to expand and abut the vessel wall 232 and/or graft 230.

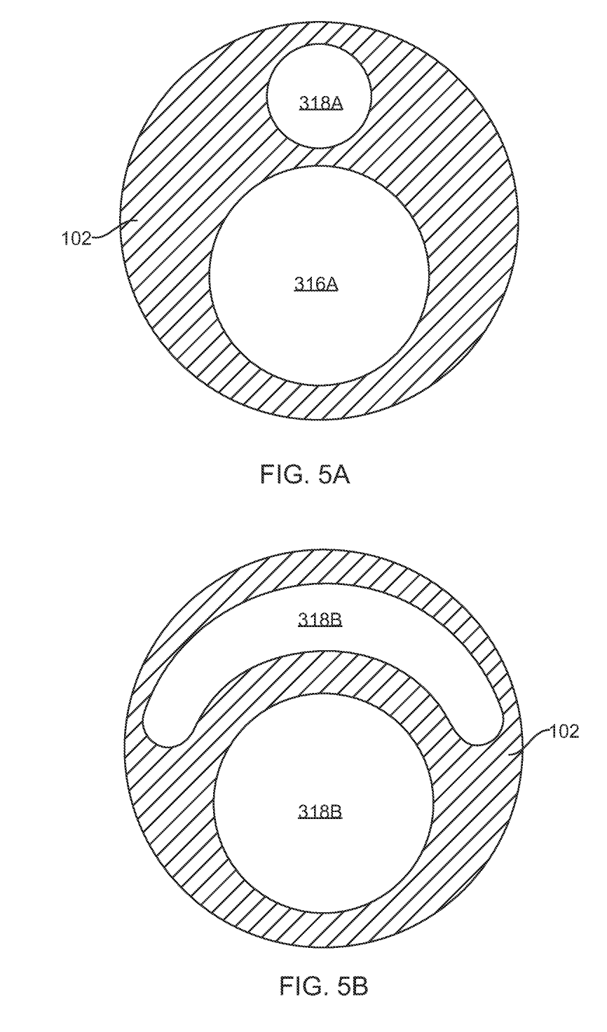

First lumen 316 and second lumen 318 are thus in a side-by-side arrangement through the length of the catheter, and may each have any suitable cross-section. For example, FIG. 5A is a cross-sectional view of endostapler delivery system 100 in accordance with one embodiment in which both first lumen 316A and second lumen 318A have circular or elliptical cross-sections. First lumen 316 of catheter shaft 102 is of a sufficient size to accommodate a stapling device. For example, a conventional stapling typically has a profile or an outer diameter of approximately 4 mm-5 mm (12-15 French units) and thus the diameter of first lumen 316 of catheter shaft 102 should be of a slightly larger size in order to ensure that a conventional stapling device can be advanced through catheter shaft 102. However, second lumen 318 of catheter shaft 102 is relatively smaller than first lumen 316 because second lumen 318 must only be of a sufficient size to accommodate the biasing mechanism, including unexpanded biasing cage 110. It is desirable to keep second lumen 318 as small as possible in order to minimize the outer diameter of catheter shaft 102, thus minimizing the size of endostapler delivery system 100 such that endostapler delivery system 100 may fit within relatively small vessels. The outer diameter of the catheter shaft may be approximately 3 mm-8 mm.

Other embodiments of catheter shaft 102 may have first lumen 316 and second lumen 318 in other dual lumen arrangements, such as a kidney or arc-shaped second lumen above a circular first lumen as shown in FIG. 5B. FIG. 5B is a cross-sectional view of the endostapler delivery system illustrated in FIG. 1 in accordance with another embodiment. Another alternative dual lumen arrangement is a crescent-shaped second lumen above a circular first lumen (not illustrated). As described above, the only limitation on the cross-sectional shapes of first lumen 316 and second lumen 318 is that first lumen 316 must be a sufficient size to accommodate a stapling device and second lumen 318 must be of a sufficient size to accommodate the biasing mechanism.

While not shown in any of the figures, the use of an outer cover, catheter outer sheath may be employed to provide a continuous smooth and slick (e.g., lubricious hydrophilic coating coated) surface to facilitate easy introduction of the catheter into the patient. Once the end of the catheter has been positioned near the delivery location, the outer cover is drawn back, either by the closing of a gap at the handle, or by splitting the outer sheath and having at least a proximal portion of it constructed as a peel away type sheath.

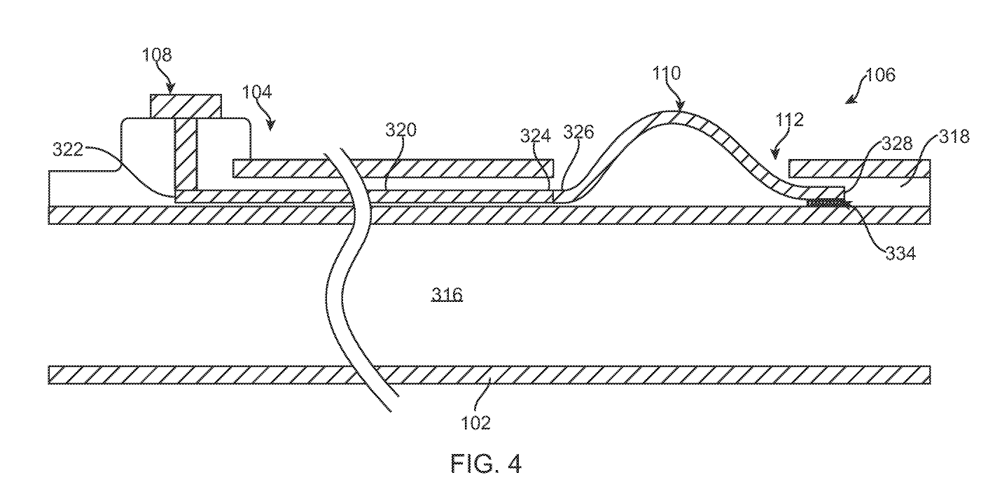

Referring now to FIGS. 3-4, biasing cage 110 is movable from an unexpanded position (shown in FIG. 3) to an expanded position (shown in FIG. 4). In the unexpanded position, biasing cage 110 is relatively straight in order to minimize the delivery profile as endostapler delivery system 100 is advanced to a position within graft 230. Further, in the unexpanded position, biasing cage 110 is completely housed within second lumen 318. Biasing cage 110 is then expanded via actuator 108 to the expanded position shown in FIG. 4, as well as FIGS. 1, 2 and 6. In the expanded position, biasing cage 110 assumes a dome or semi-circular shape extending outside of catheter shaft 102 via side recess or port 112 such that biasing cage 110 abuts the vessel wall 232 and/or graft 230. Thus, the height of the expanded biasing cage 110 must be sufficient to enable the biasing cage 110 to abut the vessel wall 232 and/or graft 230. For example, a target vessel lumen may be approximately 36 mm in diameter. Accordingly, if the outer diameter of catheter shaft 102 is approximately 3 mm-8 mm, the deployment height of the expanded biasing cage (that is, the height of the dome or semi-circular shape extending outside of catheter shaft 102) should be approximately 12 mm-30 mm or of a slightly larger size in order to ensure that the expanded biasing cage 110 abuts the vessel wall 232 and/or graft 230.

As shown in FIGS. 3 and 4, to expand biasing cage 110, actuator 108 may be a turning or push-pull actuator (i.e., a knob or handle) that is attached or connected to a rod 320 which extends through second lumen 318. Rod 320 has a proximal end 322 and a distal end 324, the proximal end 322 being connected to actuator 108 and the distal end 324 being connected to a proximal end 326 of biasing cage 110. A distal end 328 of biasing cage 110 is fixed via a connection 334 to catheter shaft 102. When actuator 108 is operated (i.e., manually turned or pushed), rod 320 is advanced through second lumen 318 of catheter shaft 102. Since distal end 328 of biasing cage 110 is fixed, biasing cage 110 expands or deploys to the expanded dome or semi-circular shape when the material of biasing cage 110 radially expands via side recess or port 112. In another embodiment, biasing cage 110 may extend through the entire second lumen 318 of catheter 102 such that the proximal end 326 of the biasing cage 110 is connected to the actuator 108, thus eliminating the need for rod 320.

Distal end 328 of biasing cage 110 may be attached to catheter shaft 102 in any suitable manner known in the art. For example, connection 334 may be formed by welding, such as by resistance welding, friction welding, laser welding or another form of welding such that no additional materials are used to connect biasing cage 110 to catheter shaft 102. Alternatively, biasing cage 110 and catheter shaft 102 can be connected by soldering, by the use of an adhesive, by the addition of a connecting element there between, or by another mechanical method.

In order to expand or deploy biasing cage 110, endostapler delivery system 100 must be tracked to and properly positioned at implanted endoluminal graft 230. In general, a guidewire (not shown) is introduced into the target vessel. Endostapler delivery system 100 is then tracked over the guidewire such that the exit port 107 is adjacent to the implanted endoluminal graft 230. Once endostapler delivery system 100 is in place as desired, the guidewire may be removed and a conventional stapling device is inserted through first lumen 316 and exit port 107 of catheter shaft 102 and tracked to a position in which the stapling device is adjacent a receiving area of the vessel wall 232 and/or graft 230 where a staple is to be fired. With the guidewire removed, endostapler delivery catheter acts as a guide catheter for tracking the conventional stapling device to the site of the implanted endoluminal graft 230. Alternatively, if the stapling device is an over the wire type device, the guidewire may be left in place within endostapler delivery system 100 and the stapling device may inserted through catheter shaft 102 and tracked over the guidewire. Alternately, the endostapler delivery catheter can be constructed with an additional lumen for a guide wire.

Once the stapling device is in place (that is, adjacent a receiving area of the vessel wall 232 and/or graft 230 where a staple is to be fired), biasing cage 110 may be expanded or deployed in order to maintain the desired position. Expansion of biasing cage 110 pushes the stapling portion of the stapling device against the vessel wall 232 and/or graft 230 where a staple is to be fired. When the staple is fired from the stapling device, biasing cage 110 remains expanded so that it prevents the stapling device from moving during the firing of the staple. Following each staple deployment, biasing cage 110 may be partially or fully collapsed to the unexpanded position. The stapling device is rotated to a second position in preparation for the firing of a second or subsequent staple, and the process is repeated to deploy the next staple. Prior to firing the second or subsequent staple, biasing cage 110 is expanded to place the stapling portion of the stapling device in position in preparation for firing. Once all the staples have been delivered and graft 230 is secured as desired, biasing cage 110 is fully collapsed to the unexpanded position. The stapling device and endostapler delivery system 100 are retracted and removed from the patient. Although methods of using specific embodiments are described herein for securing an endoluminal graft to a vessel wall, it will be apparent to those of ordinary skill in the art that such embodiments may also be utilized for securing extraluminal or transluminal grafts to a vessel wall.

Ribbons 114 of biasing cage 110 are preferably constructed of biocompatible materials having good mechanical strength. For example, non-exhaustive examples of metallic materials for ribbons 114 are stainless steel, cobalt based alloys (605L, MP35N), titanium, tantalum, tungsten based alloys, superelastic nickel-titanium alloy, other biocompatible metals, thermoplastic polymers, or combinations of any of these.

The catheter shaft may be an extruded multi-lumen shaft formed of any suitable flexible polymeric material. Non-exhaustive examples of material for the catheter shaft are polyethylene terephalate (PET), nylon, polyethylene, PEBAX, or combinations of any of these, either blended or co-extruded. Optionally, a portion of the catheter shaft may be formed as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. In an embodiment, the proximal portion of the catheter shaft may in some instances be formed from a reinforced polymeric tube, for example, as shown and described in U.S. Pat. No. 5,827,242 to Follmer et al. which is incorporated by reference herein in its entirety. The catheter shaft may have any suitable working length, for example, 550 mm-650 mm, in order to extend to a target location where a staple is to be fired.

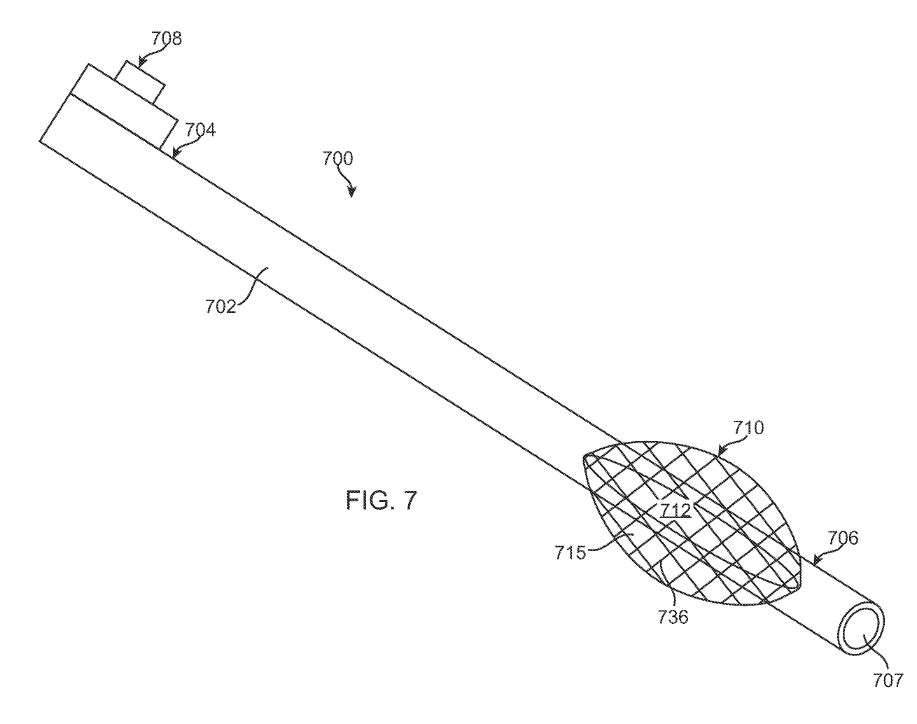

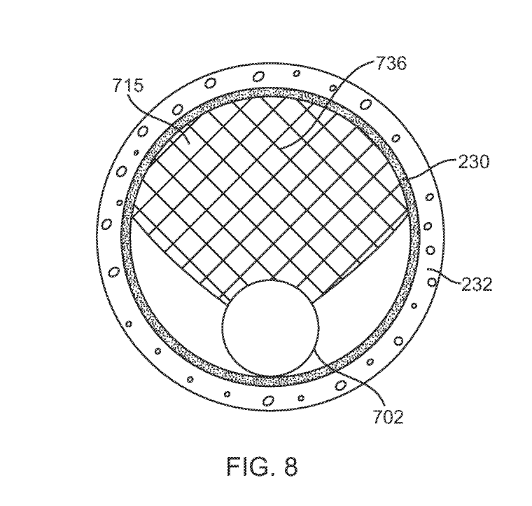

As previously discussed, embodiments described relate to a biasing mechanism to ensure that the stapling portion of the stapling device is secure against a vessel wall and/or graft. Another embodiment of a biasing device which may be utilized for this purpose is shown in FIGS. 7-10. Referring to FIGS. 7 and 8, an endostapler delivery system 700 includes a catheter shaft 702 having an expandable biasing cage 710 at the distal portion thereof. FIG. 7 is a schematic isometric view of endostapler delivery system 700, and FIG. 8 is a front view of the endostapler delivery system 700 utilized within a vessel for attaching an endoluminal graft to a vessel wall. Catheter shaft 702 includes a proximal portion 704 and a distal portion 706, wherein distal portion 706 includes an exit port 707. A side recess or port 712 is provided at the distal portion 706 of catheter shaft 702 for exposing an expandable biasing cage 710. An actuator 708 is provided at the proximal portion 704 of catheter shaft 702 for expanding biasing cage 710 to a dome or semi-circular shape. Biasing cage 710 is expanded to the dome or semi-circular shape in situ in order to ensure that a stapling device inserted through catheter shaft 702 abuts a vessel and/or graft. The stapling device may be any conventional stapling device capable of securing graft 230 to vessel wall 232.

Biasing cage 710 includes a braided structure or mesh 736. Open spaces 715 disposed within mesh 736 when biasing cage 710 is expanded allow blood or other fluid to flow through the vessel during the stapling procedure. The braided structure or mesh 736 has sufficient mechanical strength to offset or counter forces generated by a stapling device when the stapling device is utilized in securing endovascular graft 230 to a vessel wall 232 of a body lumen. More particularly, biasing cage 710 may be expanded prior to the firing of a staple. Expanding biasing cage 710 forces the stapling device against a receiving area of a vessel wall 232 and/or graft 230 where a staple is to be fired. Preferably, the receiving area of the vessel wall 232 and/or graft 230 is positioned on the opposite side of the vessel than biasing cage 710. In addition to placing the stapling device immediately adjacent to the receiving area of the vessel wall 232 and/or graft 230, biasing cage 710 also assists with preventing the stapling device from moving during the firing of the staple.

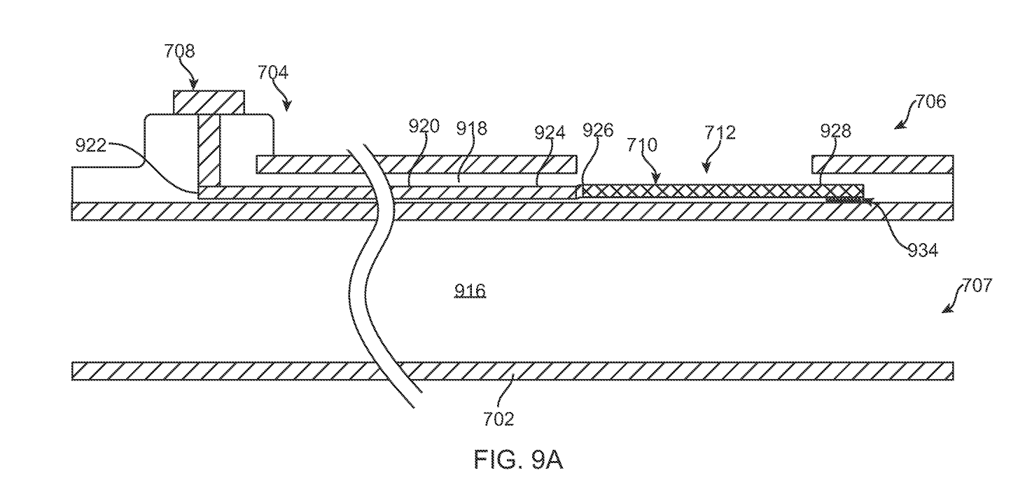

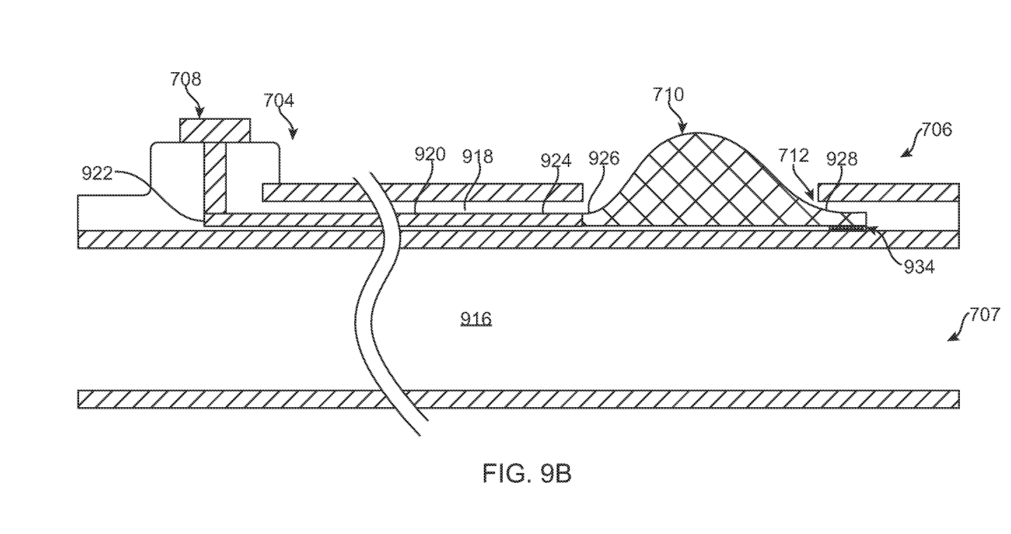

As shown in FIG. 9A, catheter shaft 702 is a multi-lumen catheter. FIG. 9A is a sectional side view of the endostapler delivery system illustrated in FIG. 7. Catheter shaft 702 includes a first lumen 916 extending along the entire length thereof for receiving a stapling device. First lumen 916 is open-ended such that it is in fluid communication with exit port 707 such that stapling device may exit out of the exit port 707 of catheter shaft 702. However, as will be explained in greater detail herein, alternatively the first lumen may be closed-ended but in fluid communication with an exit port located in the side of the catheter shaft such that a side-firing stapling device may be used. Catheter shaft 702 also includes a second lumen 918 that extends from the proximal portion 704 to the distal portion 706 of catheter shaft 702 for housing the biasing mechanism, including biasing cage 710. Second lumen 918 is parallel and adjacent to first lumen 916. Second lumen 918 is closed-ended but in fluid communication with side recess or port 712 provided at the distal portion 706 of catheter shaft 702. Side recess or port 712 allows biasing cage 710 to expand and abut the vessel wall 232 and/or graft 230. As described above with respect to previous embodiments, first lumen 916 of catheter shaft 702 is of a sufficient size to accommodate a stapling device and second lumen 918 is of a sufficient size to accommodate the biasing mechanism, including biasing cage 710. First lumen 916 and second lumen 918 may each have any suitable cross-section such as those described with respect to previous embodiments.

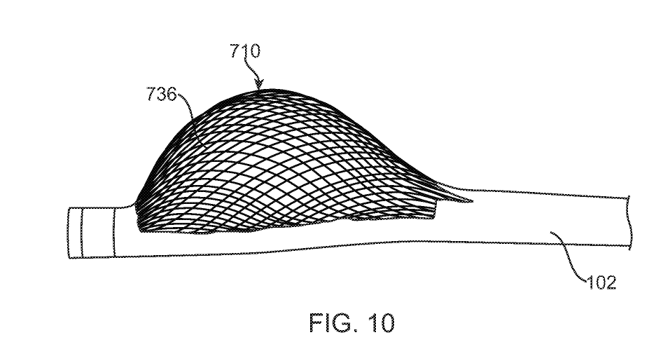

Referring now to FIGS. 9A-9B, biasing cage 710 is movable from an unexpanded position (shown in FIG. 9A) to an expanded position (shown in FIG. 9B). In the unexpanded position, biasing cage 710 is relatively straight in order to minimize the delivery profile as endostapler delivery system 700 is advanced to graft 230. Further, in the unexpanded position, biasing cage 710 is completely housed with second lumen 918. Biasing cage 710 is then expanded via actuator 708 to the expanded position shown in FIGS. 9B and 10. In the expanded position, biasing cage 710 assumes a dome or semi-circular shape extending outside of catheter shaft 702 via side recess or port 712 such that biasing cage 710 abuts the vessel wall 232 and/or graft 230. Thus, the height of the expanded biasing cage 710 must be sufficient to enable the biasing cage 710 to abut the vessel wall 232 and/or graft 230. In order to expand biasing cage 710, actuator 708 may be a rotational (to be turned) or push-pull actuator (i.e., a knob or handle) that is attached or connected to a rod 920 which extends through second lumen 918. Rod 920 includes a proximal end 922 and a distal end 924, the proximal end 922 being connected to actuator 708 and the distal end 924 being connected to a proximal end 926 of biasing cage 710. A distal end 928 of biasing cage 710 is fixed via a connection 934 to catheter shaft 702. Distal end 928 of biasing cage 710 may be attached to catheter shaft 702 in any suitable manner known in the art as described above with respect to previous embodiments. When actuator 708 is operated (i.e., manually, turned, rotated, or pushed), rod 920 is advanced through second lumen 918 of catheter shaft 702. Since distal end 928 of biasing cage 710 is fixed, biasing cage 710 expands or deploys to the expanded dome or semi-circular shape when the material of biasing cage 710 radially expands via side recess or port 712. In another embodiment, biasing cage 710 may extend through the entire second lumen 918 of catheter shaft 702 such that the proximal end 926 of the biasing cage 710 is connected to the actuator 708, thus eliminating the need for rod 920.

Mesh 736 (shown in FIG. 10) of biasing cage 710 is preferably constructed of implantable polymeric or metallic materials having good mechanical strength. Non-exhaustive examples of polymeric materials for mesh 736 are polyurethane, polyethylene terephalate (PET), nylon, polyethylene, PEBAX, or combinations of any of these, either blended or co-extruded. Non-exhaustive examples of metallic materials for mesh 736 are stainless steel, cobalt based alloys (605L, MP35N), titanium, tantalum, superelastic nickel-titanium alloy, or combinations of any of these.

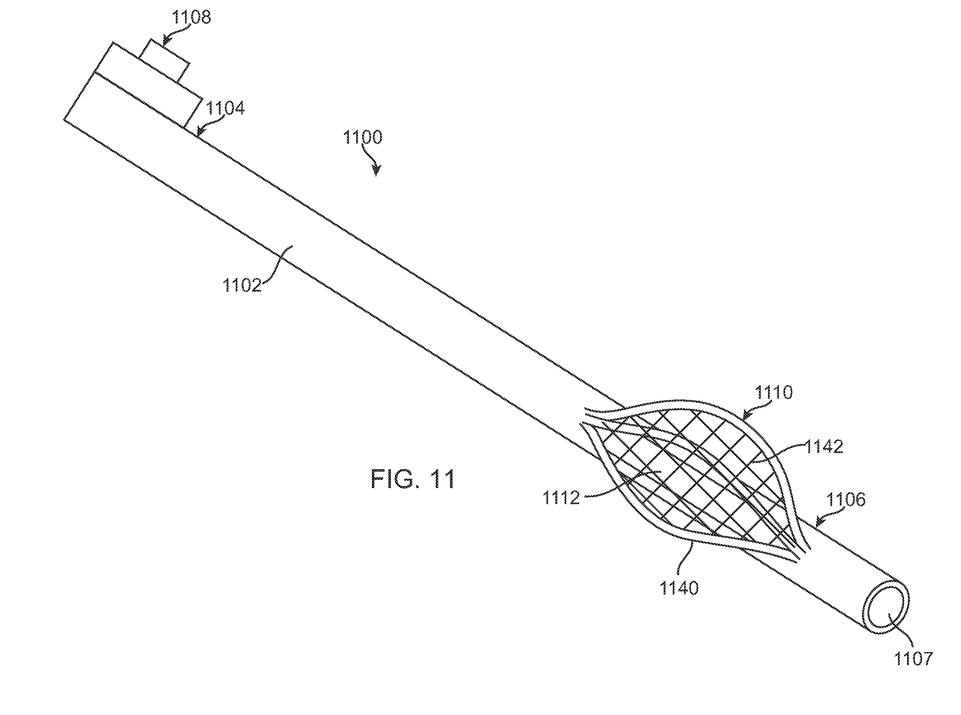

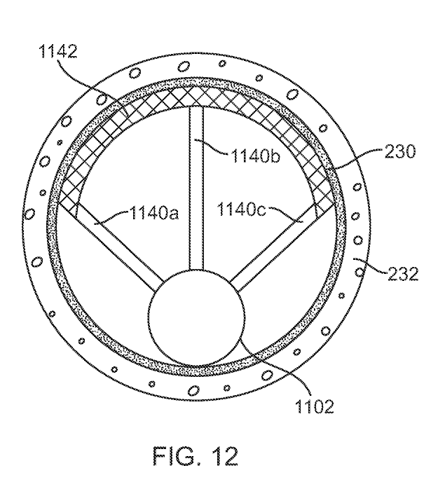

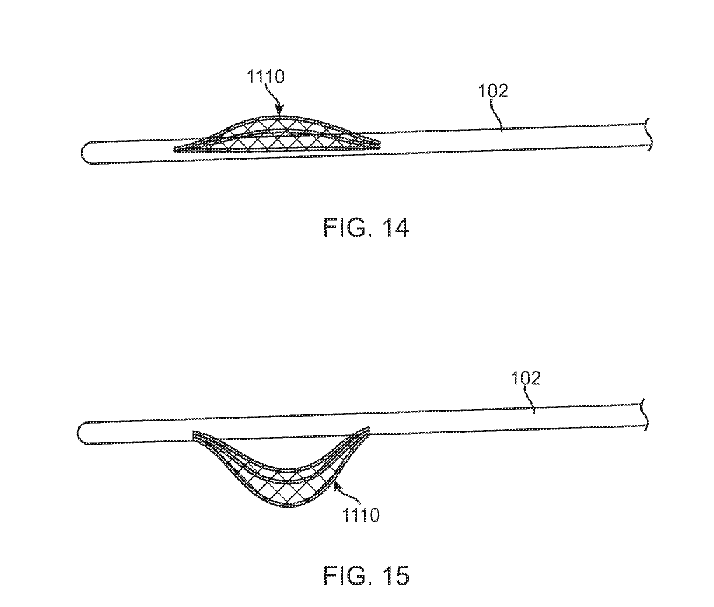

As previously discussed, the embodiments described relate to a biasing mechanism to ensure that the stapling portion of the stapling device is secure (anchored) against a vessel wall and/or graft. Another embodiment of a biasing device which may be utilized for this purpose is shown in FIGS. 11-15. Referring to FIGS. 11 and 12, an endostapler delivery system 1100 includes a catheter shaft 1102 having an expandable biasing cage 1110 at a distal portion thereof. FIG. 11 is a schematic isometric view of endostapler delivery system 1100, and FIG. 12 is a front view of the endostapler delivery system 1100 utilized within a vessel for attaching an endoluminal graft 230 to a vessel wall 232. Catheter shaft 1102 includes a proximal portion 1104 and a distal portion 1106, wherein distal portion 1106 includes an exit port 1107. A side recess or port 1112 is provided at the distal portion 1106 of catheter shaft 1102 for exposing an expandable biasing cage 1110. An actuator 1108 is provided at the proximal portion 1104 of catheter shaft 1102 for expanding biasing cage 1110 to a dome or semi-circular shape. Biasing cage 1110 is expanded to the dome or semi-circular shape in situ in order to ensure that a stapling device inserted through catheter shaft 1102 abuts a vessel wall 232 and/or a graft 230. The stapling device may be any conventional stapling device capable of securing graft 230 to vessel wall 232.

Biasing cage 1110 includes a plurality of ribbons or strands 1140 that extend generally parallel to the blood flow when expanded, and includes a braided structure or mesh 1142 placed over the plurality of ribbons 1140. Biasing cage 1110 does not block or occlude a vessel and thus allows blood or other fluid to flow there through during the stapling procedure. In one example shown in FIGS. 11-15, biasing cage 1110 includes three ribbons 1140a, 1140b, and 1140c. However, one of ordinary skill in the art will appreciate that biasing cage 1110 may include any number of ribbons or strands. For example, biasing cage 1110 may include between two and five ribbons or strands that extend generally parallel to the blood flow when expanded. In this embodiment, the plurality of ribbons 1140 have sufficient mechanical strength to offset or counter forces generated by a stapling device when the stapling device is utilized in securing endovascular graft 230 to a vessel wall 232 of a body lumen while mesh 1142 provides atraumatic gentle contact with a vessel wall. More particularly, biasing cage 1110 may be expanded prior to the firing of a staple. Expanding biasing cage 1110 forces the stapling device against a receiving area of a vessel wall 232 and/or graft 230 where a staple is to be fired. Preferably, the receiving area of the vessel wall 232 and/or graft 230 is positioned on the opposite side of the vessel than biasing cage 1110. In addition to placing the stapling device immediately adjacent to the receiving area of the vessel wall 232 and/or graft 230, biasing cage 1110 also assists with preventing the stapling device from moving during the firing of the staple.

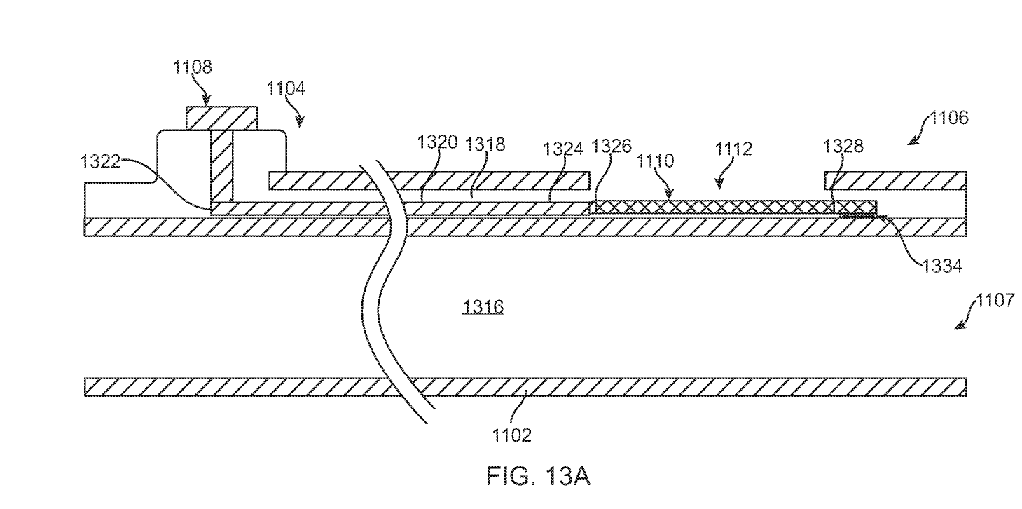

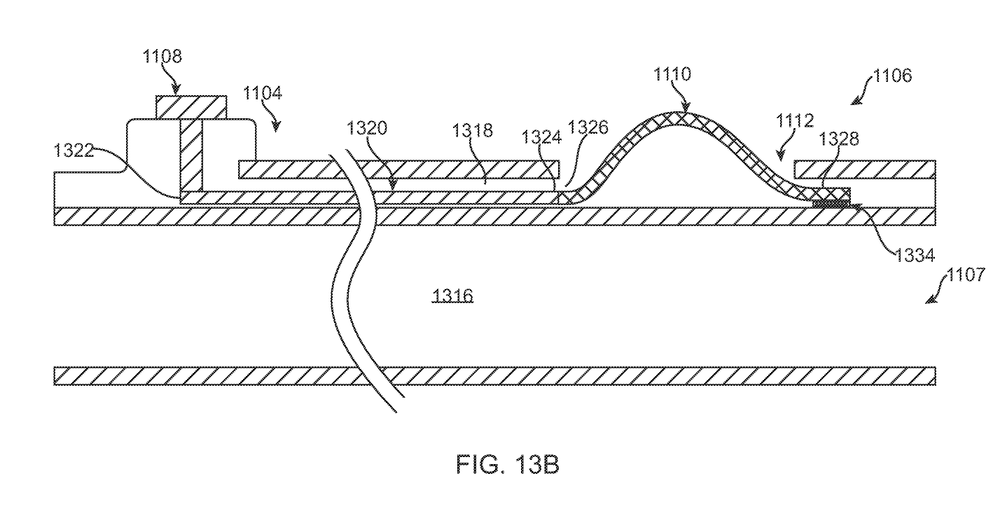

As shown in FIG. 13A, catheter shaft 1102 is a multi-lumen catheter. FIG. 13A is a sectional side view of the endostapler delivery system illustrated in FIG. 11. Catheter shaft 1102 includes a first lumen 1316 extending along the entire length thereof for receiving a stapling device. First lumen 1316 is open-ended and in fluid communication with exit port 1107 such that the stapling device may exit out of the exit port 1107 of catheter shaft 1102. However, as will be explained in greater detail herein, alternatively the first lumen may be closed-ended but in fluid communication with an exit port located in the side of the catheter shaft such that a side-firing stapling device may be used. Catheter shaft 1102 also includes a second lumen 1318 that extends from the proximal portion 1104 to the distal portion 1106 of catheter shaft 1102 for housing the biasing mechanism, including biasing cage 1110. Second lumen 1318 is parallel and adjacent to first lumen 1316. Second lumen 1318 is closed-ended but in fluid communication with side recess or port 1112 provided at the distal portion 1106 of catheter shaft 1102. Side recess or port 1112 allows biasing cage 1110 to expand and abut the vessel wall 232 and/or graft 230. As described above with respect to previous embodiments, first lumen 1316 of catheter shaft 1102 is of a sufficient size to accommodate a stapling device and second lumen 1318 is of a sufficient size to accommodate the biasing mechanism, including biasing cage 1110. First lumen 1316 and second lumen 1318 may each have any suitable cross-section such as those described with respect to previous embodiments.

Referring now to FIGS. 13A-13B, biasing cage 1110 is movable from an unexpanded position (shown in FIG. 13A) to an expanded position (shown in FIG. 13B). In the unexpanded position, biasing cage 1110 is relatively straight in order to minimize the delivery profile as endostapler delivery system 1100 is advanced to graft 230. Further, in the unexpanded position, biasing cage 1110 is completely housed with second lumen 1318. Biasing cage 1110 is then expanded via actuator 1108 to the expanded position shown in FIGS. 13B and 14-15. In the expanded position, biasing cage 1110 assumes a dome or semi-circular shape extending outside of catheter shaft 1102 via side recess or port 1112 such that biasing cage 1110 abuts the vessel wall 232 and/or graft 230. Thus, the height of the expanded biasing cage 1110 must be sufficient to enable the biasing cage 1110 to abut the vessel wall 232 and/or graft 230. To expand biasing cage 1110, actuator 1108 may be a rotational (to be turned) or push-pull actuator (i.e., a knob or handle) that is attached or connected to a rod 1320 which extends through second lumen 1318. Rod 1320 has a proximal end 1322 and a distal end 1324, the proximal end 1322 being connected to actuator 1108 and the distal end 1324 being connected to a proximal end 1326 of biasing cage 1110. A distal end 1328 of biasing cage 1110 is fixed via a connection 1334 to catheter shaft 1102. Distal end 1328 of biasing cage 1110 may be attached to catheter shaft 1102 in any suitable manner known in the art as described above with respect to previous embodiments. When actuator 1108 is operated (i.e., manually, turned, rotated, or pushed), rod 1320 is advanced through second lumen 1318 of catheter shaft 1102. Since second distal end 1328 of biasing cage 1110 is fixed, biasing cage 1110 expands or deploys to the expanded dome or semi-circular shape when the material of biasing cage 1110 radially expands via side recess or port 1112. In another embodiment, biasing cage 1110 may extend through the entire second lumen 1318 of catheter shaft 1102 such that the proximal end 1326 of the biasing cage 1110 is connected to the actuator 1108, thus eliminating the need for rod 1320.

Mesh 1142 is positioned or superimposed over ribbons 1140 to form biasing cage 1110. Mesh 1142 of biasing cage 1110 provides atraumatic gentle contact with the vessel wall and thus is preferably constructed of a flexible implantable polymeric material. Non-exhaustive examples of polymeric materials for mesh 1142 are polyurethane, polyethylene terephalate (PET), nylon, polyethylene, PEBAX, or combinations of any of these, either blended or co-extruded. Ribbons 1140 have sufficient mechanical strength to offset or counter forces generated by a stapling device and thus are preferably constructed from an implantable metallic material having good mechanical strength. Non-exhaustive examples of metallic materials for ribbons 1140 are stainless steel, cobalt based alloys (605L, MP35N), titanium, tantalum, superelastic nickel-titanium alloy, or combinations of any of these.

Biasing cage 1110 having a combination of a plurality of ribbons or strands 1140 and a braided structure or mesh 1142 would have an advantage of a smaller delivery profile. Ribbons 1140 act as the structural element in that they provide the majority of the structural support needed to assure catheter contact with the vessel wall. Ribbons 1140 can be constructed with a narrower cross sectional configuration to minimize catheter crossing profile, as the adjacent mesh structure 1142 will distribute the force exerted over a larger area than just the surface of the ribbons and as such will provide a combined element that provides atraumatic contact with the vessel wall. The general understood means of forming such shape memory ribbons would be used to shape the ribbon to pre-set shape expanded predetermined diameter. In operation, a push pull and/or screw actuation mechanism would then be used for deployment.

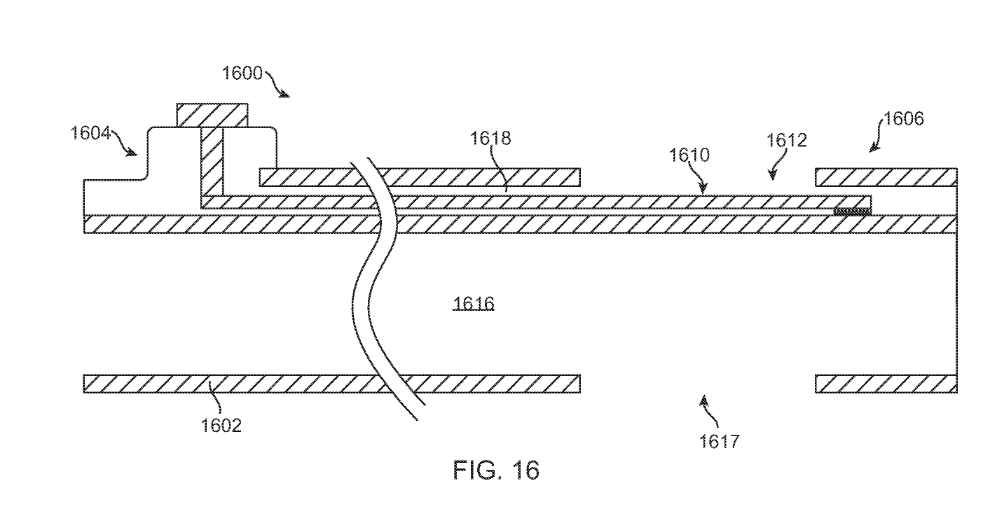

As previously described, the first lumen of the catheter shaft that receives the stapling device may be open-ended and in fluid communication with an exit port such that the stapling device may exit out of the distal open-ended exit port. Alternatively, the first lumen of the catheter shaft may be closed-ended but in fluid communication with an exit port located in the side of the catheter shaft such that a side-firing stapling device may be utilized. For example, as shown in FIG. 16, catheter shaft 1602 is a multi-lumen catheter including a first lumen 1616 extending along the entire length thereof for receiving a stapling device. First lumen 316 is closed-ended but in fluid communication with side exit port 1617 of the catheter such that a side-firing stapling device may exit out of the side exit port 1617 at the distal portion 1606 of catheter shaft 102. Similar to previously described embodiments, catheter shaft 1602 also includes a second lumen 1618 that extends from the proximal portion 1604 to the distal portion 1606 of catheter shaft 1602 for housing the biasing mechanism, including biasing cage 1610. Second lumen 1618 is parallel and adjacent to first lumen 1616. Second lumen 1618 is closed-ended but in fluid communication with side recess or port 1612 provided at the distal portion 1606 of catheter shaft 1602 to allow biasing cage 1610 to expand and abut the vessel wall and/or graft. Distal side exit port 1617 is located directly across from (on the opposite side of the catheter shaft) side recess or port 1612 so that a staple is fired directly opposite from the approximate centerline of an expanded portion of the biasing mechanism (biasing cage 1610).

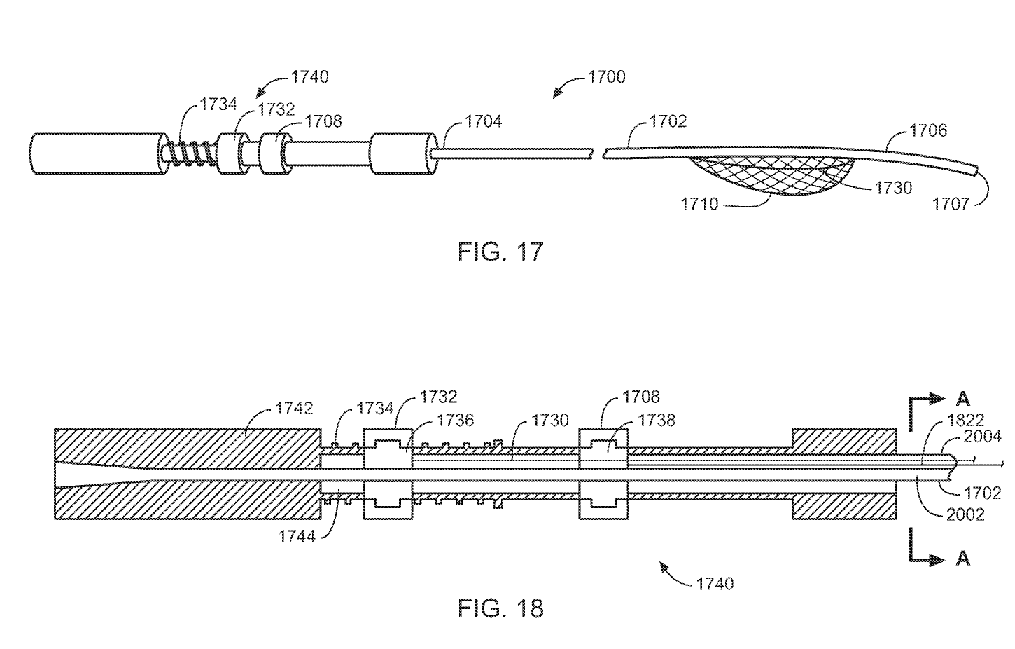

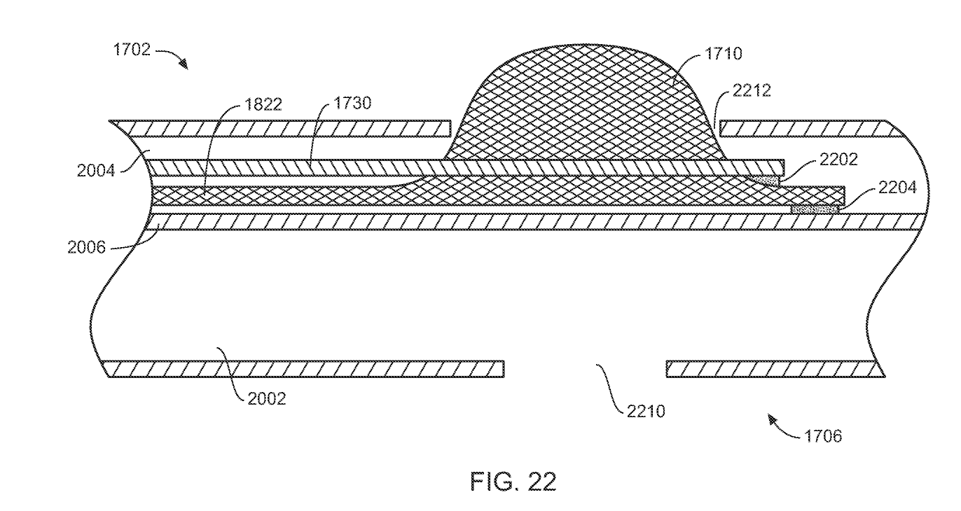

FIGS. 17-22 show another embodiment of an endostapler delivery system 1700. Endostapler delivery system 1700 includes a catheter shaft 1702 having an expandable biasing cage 1710 at the distal portion thereof. Catheter shaft 1702 includes a proximal portion 1704 and a distal portion 1706. A distal exit port 1707 may be included at a distal end of distal portion 1706. A side recess or port 2212 (see FIG. 22) is provided at the distal portion 1706 of catheter shaft 1702 for exposing an expandable biasing cage 1710. A handle 1740 is provided at the proximal portion 1704 of catheter shaft 1702. Handle 1740 includes an actuator 1708 for expanding biasing cage 1710 to a dome or semi-circular shape. Biasing cage 1710 is expanded to the dome or semi-circular shape in situ in order to ensure that a stapling device inserted through catheter shaft 1702 abuts a vessel and/or graft. The stapling device may be any conventional stapling device capable of securing a graft to a vessel wall, as described above. Biasing cage 1710 in this embodiment is the same structure as the embodiment described above with respect to FIGS. 7-10, but may also be the structure described with respect to the other embodiments herein.

Endostapler delivery system 1700 also includes a steering wire 1730. Steering wire 1730 is coupled to a distal portion of biasing cage 1710, as shown in FIG. 22. Further, handle 1740 includes a steering wire actuator 1732. In the embodiment shown in FIGS. 17-22, steering wire actuator 1732 is rotated and moves proximally along threads 1734 of handle 1740 to pull steering wire 1730 proximally. Pulling steering wire 1730 proximally causes catheter shaft 1702 to bend in order to navigate tortuous paths or to situate biasing cage 1710 and the stapler in a curved portion of a vessel. As shown in FIGS. 17, 19, and 22, steering wire 1720 extends through biasing cage 1710, but, depending on the embodiment of the biasing cage used, steering wire may extend, for example, underneath the biasing cage. As shown in FIG. 22, steering wire 1730 is coupled to biasing cage 1710 at 2202 by welding, soldering, adhesive, or other suitable means known to those skilled in the art. Further, although steering wire 1730 is shown coupled to biasing cage 1710, it may alternatively be coupled to a portion of catheter shaft 1702, for example, wall 2006 dividing the lumens of catheter shaft 1702.

FIG. 18 is a partial cross-sectional schematic illustration of handle 1740. Handle 1740 includes a body 1742 and a bore 1744 disposed therethrough. Steering wire actuator 1732 is disposed around an outside surface of body 1742 and is coupled to a steering wire follower 1736 disposed within bore 1744. The steering wire follower includes a keel or “T-tube” having lateral members that extend through one or a set of two longitudinal slots in the surface of the threads 1734. The steering wire actuator 1732 is slidably engaged with the keel or “T-tube” “T” pieces to force the axial movement of the steering wire follower 1736 as the actuator 1732 is turned along threads 1734 (an example of a “T-tube” and its engagement with a rotating handle can be seen as items 1712 or 23-7 in to Shiu U.S. Pat. No. 7,105,016 incorporated herein by reference). Steering wire follower 1736 is coupled to steering wire 1730. Steering wire 1730 may be, for example, a 0.012″ stainless steel wire. Steering wire 1730 may be coupled to steering wire follower 1736 by laser welding steering wire 1730 inside a capillary tube and embedding the capillary tube into steering wire follower 1736. Other means to couple steering wire 1730 to steering wire follower 1736 may be used as would be apparent to those skilled in the art. Steering wire actuator 1732 starts in its distal position.

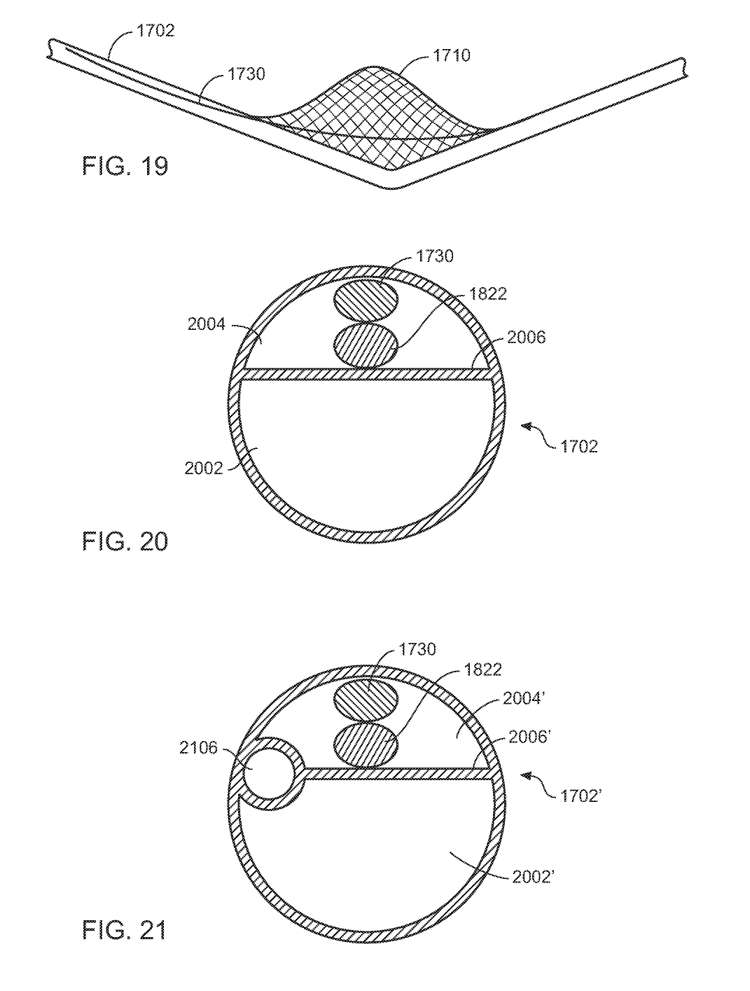

In this embodiment, steering wire actuator 1732 is rotated around body 1742, thereby moving proximally or distally along threads 1734 of body 1742. Movement of steering wire actuator 1732 causes steering wire follower 1736 and a proximal end of steering wire 1730 to move with steering wire actuator 1732. Because distal end of steering wire 1730 is fixed, moving steering wire actuator 1732 proximally transfers the force of the proximal movement to catheter shaft 1702, thereby bending catheter 1702 as shown in FIG. 19. Turning steering wire actuator 1732 in the opposite direction returns steering wire actuator to its distal position and allows catheter shaft 1702 to return to its straightened configuration.

Biasing cage actuator 1708 is also disposed around body 1742 and is coupled to a biasing cage follower 1738 disposed within bore 1744. A proximal end of a rod 1822 is coupled to biasing cage follower 1738 and a distal end of rod 1822 is coupled to biasing cage 1710 (see FIG. 22). The proximal end of rod 1822 may be coupled to biasing cage follower 1738 using adhesive, a mechanical bond, laser weld, or other suitable means known to those skilled in the art. Sliding biasing cage actuator 1708 distally causes biasing cage 1710 to expand, and sliding biasing cage actuator proximally to its original position returned biasing cage 1710 to its unexpanded configuration, as described above with respect to FIGS. 7-10. Although steering wire actuator 1732 has been shown and described as a screw-type or rotatable actuator, and biasing cage actuator has been shown and described as a sliding actuator, it would be understood by those skill in the art that any suitable actuator can be used for either or both actuators.

FIGS. 20 and 21 show embodiments of catheter shaft 1702 in cross-sections taken along line A-A of FIG. 18. FIG. 20 shows a dual lumen embodiment. In the embodiment of FIG. 21, catheter shaft 1702 is divided into a first a stapler lumen 2002 and a braid lumen 2004 by a wall 2006. Stapler lumen 2002 is used to delivery the stapler to the treatment site. Stapler lumen 2004 may also be used for other purposes, such as for a guidewire used to track the catheter to the treatment site. Braid lumen 2004 includes rod 1822 for actuating biasing cage 1710 and steering wire 1730. In the embodiment shown in FIG. 20, rod 1822 and steering wire 1730 are stacked vertically, but they could be disposed side-by-side, or in any other suitable manner known to those skilled in the art. FIG. 21 shows an alternative embodiment of a catheter shaft 1702′. Catheter shaft 1702′ is a three-lumen design, including a stapler lumen 2002′, a braid lumen 2004′ and a guidewire lumen 2106. Guidewire lumen 2106 is used for a guidewire. In other aspects, catheter shaft 1702′ is the same as catheter shaft 1702.

FIG. 22 shows a sectional side view of distal portion 1706 of catheter shaft 1702 with biasing cage 1710 in its expanded configuration expanded through side port 2212. A side port 2210 is disposed in catheter shaft 1702 opposite side port 2212 and accessible through stapler lumen 2002. The staple or clip of the stapler is actuated through side port 2210. Rod 1822 and steering wire 1730 are stacked vertically in braid lumen 2004, with a distal end of steering wire 1720 coupled to biasing cage 1710 at connection 2202 and a distal end of biasing cage is coupled to wall 2006 at connection 2204.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of that described. It will also be understood that each feature of each embodiment discussed herein, and of each reference cited herein, can be used in combination with the features of any other embodiment. All patents and publications discussed herein are incorporated by reference herein in their entirety.

What is claimed is:

1. An endostapler delivery system for delivering a stapling device through a body lumen, comprising:
a catheter shaft including a proximal portion and a distal portion, the catheter shaft defining a first lumen and a second lumen having a side exit port disposed at the distal portion of the catheter shaft, wherein the first lumen of the catheter shaft is of a sufficient size such that the stapling device may be advanced there through;
an expandable biasing cage disposed within the second lumen of the catheter shaft;
a first actuator disposed at the proximal portion of the catheter shaft, wherein the first actuator is configured to expand the biasing cage to a dome shape extending outside of the catheter shaft via the side exit port, wherein the biasing cage is configured such that it permits fluid flow past the biasing cage when configured in an expanded configuration;
a steering wire disposed within the second lumen and coupled to a distal portion of the catheter shaft; and
a second actuator disposed at the proximal portion of the catheter shaft and coupled to a proximal portion of the steering wire, wherein the second actuator is configured to bend the catheter shaft through the steering wire.

2. The endostapler delivery system of claim 1, wherein the biasing cage has an unexpanded configuration that lies completely within the second lumen of the catheter shaft.

3. The endostapler delivery system of claim 1, wherein the first and second actuators are selected from the group consisting of a sliding actuator and a turning actuator.

4. The endostapler delivery system of claim 3, wherein the first actuator is a sliding actuator and the second actuator is a turning actuator.

5. The endostapler delivery system of claim 1, further comprising:
a rod disposed in the second lumen, wherein a proximal portion of the rod is coupled to the first actuator and a distal portion of to rod is coupled to the expandable biasing cage.

6. The endostapler delivery system of claim 5, wherein the distal portion of the rod is coupled to a proximal portion of the expandable biasing cage, and wherein a distal portion of the expandable biasing cage is coupled to the distal portion of the catheter shaft such that the first actuator moves the rod distally while the distal portion of the expandable biasing cage is fixed to expand the expandable biasing cage.

7. The endostapler delivery system of claim 6, wherein a distal portion of the steering wire is coupled to the distal portion of the expandable biasing cage.

8. The endostapler delivery system of claim 1, wherein the first lumen includes an exit port disposed in the distal portion of the catheter shaft.

9. The endostapler delivery system of claim 8, wherein the exit port of the first lumen is a side exit port.

10. The endostapler delivery system of claim 9, wherein the exit port of the first lumen is located generally opposed from the side exit port of the second lumen.

11. The endostapler delivery system of claim 1, wherein first lumen is open-ended at a distal end and the exit port of the first lumen is located at the open-ended distal end of the catheter shaft.

12. The endostapler delivery system of claim 1, wherein the biasing cage is formed from a plurality of ribbons.

13. The endostapler delivery system of claim 12, wherein the plurality of ribbons are constructed from a material selected from the group consisting of stainless steel, a cobalt alloy, titanium, titanium alloys, tantalum, tantalum alloys, a nickel-titanium alloy, and tungsten alloys.

14. The endostapler delivery system of claim 1, wherein the biasing cage is formed from a mesh structure.

15. The endostapler delivery system of claim 14, wherein the mesh structure is constructed from a material selected from the group consisting of stainless steel, a cobalt alloy, titanium, titanium alloys, tantalum, tantalum alloys, a nickel-titanium alloy, and tungsten alloys.

16. The endostapler delivery system of claim 1, wherein the biasing cage is formed from a plurality of ribbons and a mesh structure disposed over the plurality of ribbons.

17. The endostapler delivery system of claim 16, wherein the plurality of ribbons are constructed from a material selected from the group consiststainless steel, a cobalt alloy, titanium, titanium alloys, tantalum, tantalum alloys, a nickel-titanium alloy, and tungsten alloys.

18. The endostapler delivery system of claim 17, wherein the mesh is constructed from a polymeric material.

19. A method of delivering a stapling device through a body lumen, the method comprising the steps:
tracking an endostapler delivery system to a target location within the body lumen, wherein the endostapler delivery system includes
a catheter shaft having a proximal portion and a distal portion, the catheter shaft defining a first lumen having a first exit port and a second lumen having a second, side exit port disposed at the distal portion of the catheter shaft,
an expandable biasing cage disposed within the second lumen of the catheter shaft,
a first actuator provided at the proximal portion of the catheter shaft;
a steering wire disposed within the second lumen and coupled to a distal portion of the catheter shaft;
a second actuator disposed at the proximal portion of the catheter shaft and coupled to a proximal portion of the steering wire;
operating the second actuator to pull the steering wire such that the catheter shaft bends;
tracking the stapling device through the first lumen of the endostapler delivery system such that the stapling device is adjacent to the target location within the body;
operating the first actuator such that the biasing cage expands to a dome shape extending outside of the catheter shaft via the second, side exit port such that the biasing cage abuts a vessel wall of the body lumen and/or a graft implanted within the body lumen, wherein the biasing cage when expanded does not block or occlude the body lumen such that blood may flow there through; and
firing a staple from the stapling device.

20. The method of claim 19, wherein the target location within the body lumen is an endovascular graft.

21. The method of claim 19, wherein the first exit port is a side port located opposite the second, side exit port.

22. The method of claim 19, wherein first lumen is open-ended at a distal end and the first exit port is located at the open-ended distal end of the catheter shaft.

23. The method of claim 19, wherein the biasing cage is formed from a plurality of ribbons that extend parallel to the blood flow such that the biasing cage when expanded does not block or occlude the body lumen such that blood may flow there through.

24. The method of claim 19, wherein the biasing cage is formed from a mesh structure such that the biasing cage when expanded does not block or occlude the body lumen such the blood may flow there through.

25. The method of claim 19, wherein the biasing cage is formed from a plurality of ribbons and a mesh structure disposed over the plurality of ribbons such that the biasing cage when expanded does not block or occlude the body lumen such that blood may flow there through.

26. The method of claim 25, wherein the mesh is constructed from a polymeric material.
